(19) United States
(12) Patent Application Publication
(10) Pub. No.: US 2026/0294274 A1
(43) Pub. Date: Oct. 1, 2026

(54) MAINSTREAM CAPNOGRAPHY

(71) Applicant: Oxlantic Medical AB, Uppsala (SE)

(72) Inventors: Anders ECKERBOM, Bromma (SE); Joakim ÖSTBLOM, Uppsala (SE)

(21) Appl. No.: 19/491,012

(22) PCT Filed: Jun. 10, 2024
(86) PCT No.: PCT/SE2024/050560

§ 371 Date: Dec. 8, 2025

(30) Foreign Application Priority Data
Jun. 20, 2023 (SE) 2350749-4

(51) Int. Cl. A61B 5/083; A61B 5/00; A61B 5/097

(52) U.S. Cl. CPC A61B 5/0836; A61B 5/097; A61B 5/6819

(57) ABSTRACT

A mainstream capnography system includes a capnography and gas delivery arrangement including an airway adapter, a flow measurement adapter-and a mainstream capnography sensor, and a portable monitor. The mainstream capnography system allows mainstream capnography measurements while administering gas to a human subject and is portable and easy to use.

### TECHNICAL FIELD

The present invention generally relates to mainstream capnography, and in particular to an airway adapter, an airway adapter arrangement, a capnography and gas delivery arrangement and a portable monitor that can be used in a mainstream capnography system.

### BACKGROUND

Capnography is the monitoring of the concentration or partial pressure of carbon dioxide (CO₂) in the respiratory gases. Its main development has been as a monitoring tool for use during anesthesia and intensive care. It is usually presented as a graph of CO₂, measured in kilopascals (kPa) or millimeters of mercury (mmHg), plotted against time. Measurements taken at the end of the exhalation are commonly referred to as end tidal CO₂ (ETCO₂).

Capnographs, i.e., CO₂ gas analyzers with a waveform display, are traditionally classified as so-called mainstream or sidestream capnographs. The fundamental difference between mainstream and sidestream capnographs is whether they divert gas from the airway for analysis. Sidestream capnographs take a small sample flow from the respiratory circuit, nostrils and/or mouth of a human subject to an adjacent instrument, in which the actual gas analysis takes place, whereas mainstream capnographs measures the CO₂ concentration directly in the respiratory circuit.

Mainstream capnographs comprise a CO₂ sensor fitted to an airway adapter connected to the respiratory circuit of a human subject. The gas to be analyzed by the CO₂ sensor is, thus, taken directly from the airway. This technology generally provides very accurate readings since the CO₂ sensor is at the actual airway.

US 2019/0298960, U.S. Pat. Nos. 5,464,982, 7,455,644, 7,445,602 and 8,915,861 disclose airway adapters for mainstream capnographs.

Sidestream capnographs pull a fraction of the human subject's exhaled air through a small tube from the respiratory circuit into a monitor comprising a CO₂ sensor. The readings obtained by sidestream capnographs are generally regarded as being less accurate than mainstream capnographs because of the variables involved with pulling the gas sample from the patient to the monitor, and into the internal CO₂ sensor.

U.S. Pat. Nos. 5,857,461, 5,335,656 and 7,383,839 disclose oral nasal cannula or airway adapters that can be used for sidestream capnographs.

There is still a need for mainstream capnographs that are portable and could be used at the home of patients for ambulatory care and diagnosis and accurately measure CO₂ in the exhalation air of patients.

### SUMMARY

It is a general objective to provide an airway adapter, an airway adapter arrangement, a capnography and gas delivery arrangement and portable monitor that can be used in a mainstream capnography system.

This and other objectives are met by embodiments as disclosed herein.

The present invention is defined in the independent claims. Further embodiments of the invention are defined in the dependent claims.

An aspect of the invention relates to an airway adapter adapted to be disposed below nostrils of a human subject. The airway adapter comprises a gas case comprising multiple gas outlets arranged to be positioned in vicinity of the nostrils of the human subject when the airway adapter is attached on a face of the human subject. The airway adapter also comprises a gas delivery tube in fluid communication with the gas case and a prong case comprising at least one nasal prong arranged to collect nasally exhaled breath from the human subject. The airway adapter also comprises an airway case defining an airway passage in fluid communication with the prong case. The airway case comprises a first light window and a second light window into the airway passage. The airway adapter is connectable to a mainstream capnography sensor comprising at least one light source and at least one light detector to position the mainstream capnography sensor onto the airway adapter to align the at last one light source with the first light window and align the at least one light detector with the second light window.

Another aspect of the invention relates to an airway adapter arrangement comprising an airway adapter according to above and a flow measurement adapter attached to the gas delivery tube. The flow measurement adapter comprises an inlet port connectable to a gas source through a gas forwarding tube. The flow measurement adapter also comprises a flow channel in fluid communication with the gas delivery tube and the inlet port and comprising a flow restriction. The flow measurement adapter further comprises a first pressure measuring port in fluid communication with the flow channel at a position upstream of the flow restriction and a second pressure measuring port in fluid communication with the flow channel downstream of the flow restriction.

A further aspect of the invention relates to a capnography and gas delivery arrangement comprising an airway adapter according to above or an airway adapter arrangement according to above and a mainstream capnography sensor. The mainstream capnography sensor comprises a sensor housing comprising a first light window, a second light window, at least one light source arranged in the sensor housing to emit infrared (IR) light through the first light window and at least one light detector arranged in the sensor housing to detect IR light through the second light window. The mainstream capnography sensor also comprises a sensor cable attached to the sensor housing and in electrical communication with the at least one light source and the at least one light detector. The first light window of the sensor housing is aligned with the first light window of the airway case and the second light window of the sensor housing is aligned with the second light window of the airway case when the mainstream capnography sensor is connected to the airway adapter.

Yet another aspect of the invention relates to a portable monitor for monitoring a spontaneously breathing human subject. The portable monitor comprises a sensor port connectable to a sensor cable of a mainstream capnography sensor and an adapter receptacle connectable to a flow measurement adapter. The portable monitor comprises a first pressure port configured to be in fluid communication with a gas flow through the flow measurement adapter upstream of a flow restriction and a second pressure port configured to be in fluid communication with the gas flow through the flow measurement adapter downstream of the flow restriction. The portable monitor further comprises a differential pressure sensor in fluid communication with the first pressure port and the second pressure port and configured to measure a pressure difference between the first pressure port and the second pressure port and generate an output signal representative of the pressure difference. The portable monitor additionally comprises a processor communicatively connected to the sensor port and the differential pressure sensor and a memory coupled to the processor and comprising instructions executable by the processor to cause the processor to process an output signal received at the sensor port and generated by the mainstream capnography sensor to generate a CO₂ parameter value representative of partial pressure of CO₂ in the exhaled air from the human subject. The processor is also caused to process the output signal from the differential pressure sensor to generate a flow rate value representative of a flow rate of the gas flow.

A further aspect of the invention relates to a mainstream capnography system comprising a capnography and gas delivery arrangement according to above and comprising an airway adapter arrangement according to above. The mainstream capnography system also comprises a portable monitor according to above.

The present invention defines a mainstream capnography system and components thereof that can be used for mainstream capnography measurements while administering gas to a human subject. The mainstream capnography system is designed to be easy to use and handle, even by non-medically trained persons. The mainstream capnography system is further portable and can thereby be used by users at home or in ambulatory care.

### BRIEF DESCRIPTION OF THE DRAWINGS

The embodiments, together with further objects and advantages thereof, may best be understood by making reference to the following description taken together with the accompanying drawings, in which:

FIG. 1 is an illustration of an airway adapter according to an embodiment in a front view;

FIG. 2 is an illustration of the airway adapter in FIG. 1 in a rear view;

FIG. 3 is a cross-sectional view of an airway adapter according to an embodiment;

FIG. 4 is an illustration of an airway adapter and a mainstream capnography sensor according to an embodiment;

FIG. 5 is an illustration of a mainstream capnography sensor according to an embodiment;

FIG. 6 is a cross-sectional view of the mainstream capnography sensor in FIG. 5;

FIG. 7 is an overview of a mainstream capnography system according to an embodiment;

FIG. 8 is an illustration of a flow measurement adapter according to an embodiment;

FIG. 9 schematically illustrates attachment of a flow measurement adapter to an adapter receptacle of a portable monitor according to an embodiment;

FIG. 10 is a block diagram of a portable monitor according to an embodiment;

FIG. 11 is an illustration of an airway adapter according to another embodiment in a front view;

FIG. 12 is an illustration of the airway adapter in FIG. 11 in a rear view;

FIG. 13 is an illustration of an airway adapter and a mainstream capnography sensor according to another embodiment;

FIG. 14 is a cross-sectional view of the mainstream capnography sensor in FIG. 13;

FIG. 15 is a partly exploded view of an airway adapter showing gas flows; and

FIG. 16 is an illustration of a capnography and gas delivery arranged attached to the face of a human subject.

### DETAILED DESCRIPTION

The present invention generally relates to mainstream capnography, and in particular an airway adapter, an airway adapter arrangement, a capnography and gas delivery arrangement and portable monitor that can be used in a mainstream capnography system.

The present invention relates to a mainstream capnography system designed to be used not only in medical facilities but also being sufficiently small to be portable to use in ambulatory care and also by patients themselves at their homes. The mainstream capnography system has been designed to include components that are connectable and function together to enable mainstream capnography measurement together with gas administration to the patient. Thus, the mainstream capnography system will not only enable accurate measurement of carbon dioxide (CO₂) in the exhalation air of a patient but may also be used to administer gas, typically oxygen (O₂) or an oxygen enriched gas mixture, to the patient while enabling a monitoring of the gas administration.

The mainstream capnography system comprises an airway adapter designed to be connectable to a mainstream capnography sensor forming a combined unit that can be attached to or on the face of the patient where the airway adapter will distribute and deliver gas from a gas source while collecting exhaled breath from the patient. The collected exhaled breath is analyzed by the mainstream capnography sensor electrically connected to a portable monitor. The portable monitor is also connectable to a flow measurement adapter arranged in fluid connection between the airway adapter and the gas source.

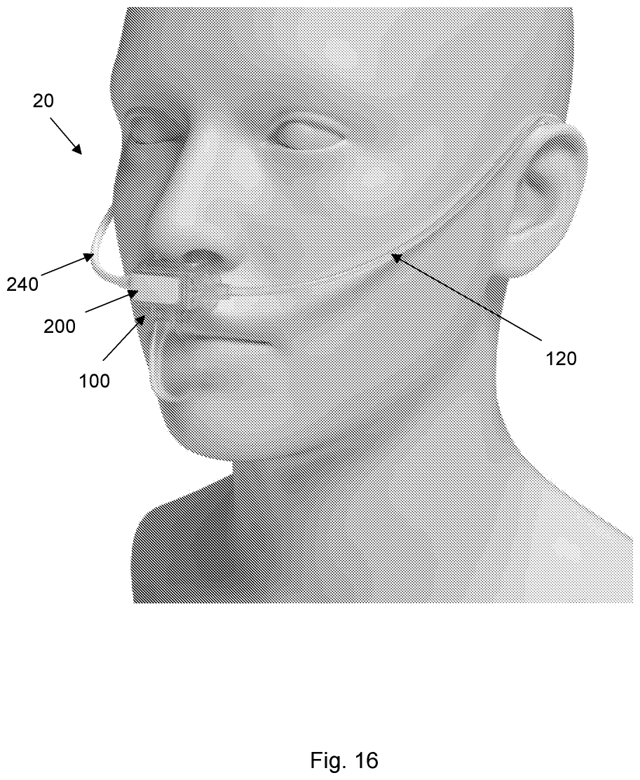

The unique design of the airway adapter and the mainstream capnography sensor and the attachment of these two units together allows an easy attachment of the airway adapter with the mainstream capnography sensor at the face of the patient without the need for dedicated attachment equipment and units by utilizing a gas delivery tube of the airway adapter and a sensor cable of the mainstream capnography sensor as attachment equipment as shown in FIG. 16. This not only maintains the airway adapter attached on the face of the patient to deliver gas and collect exhaled air but enables this with a minimum of tubing and cables.

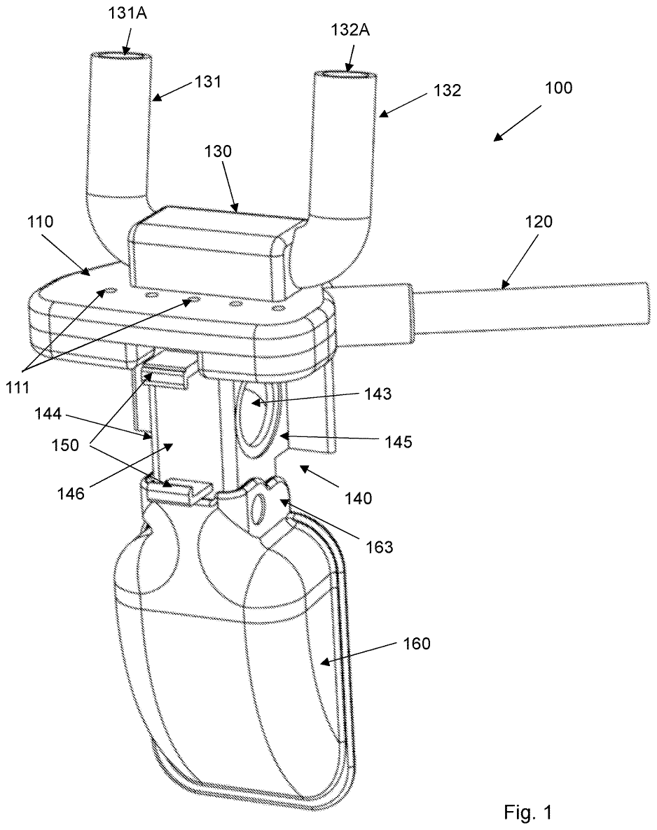

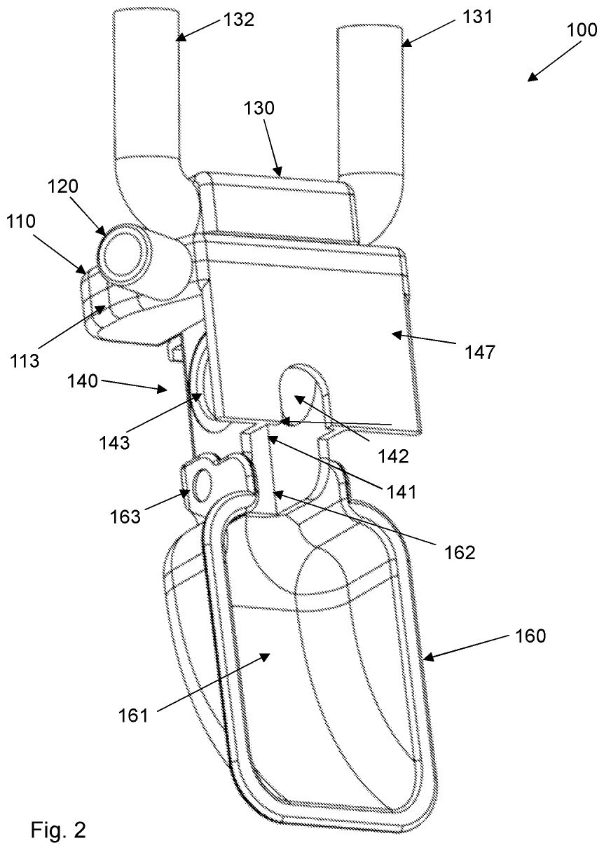

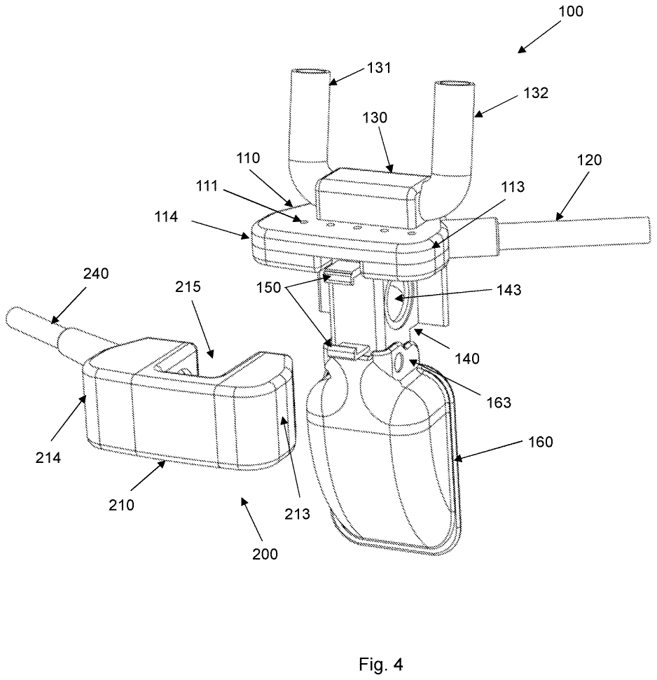

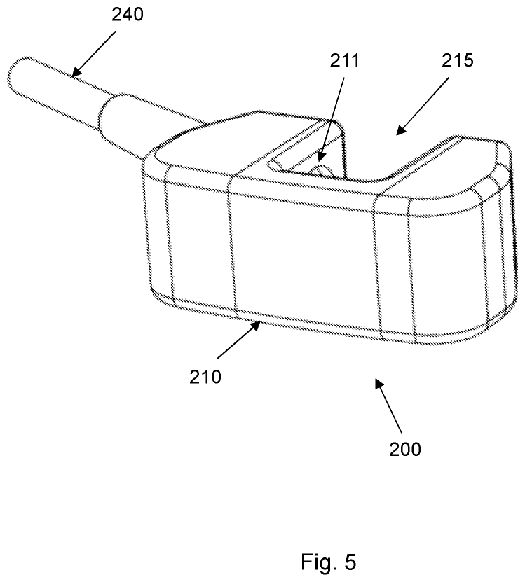

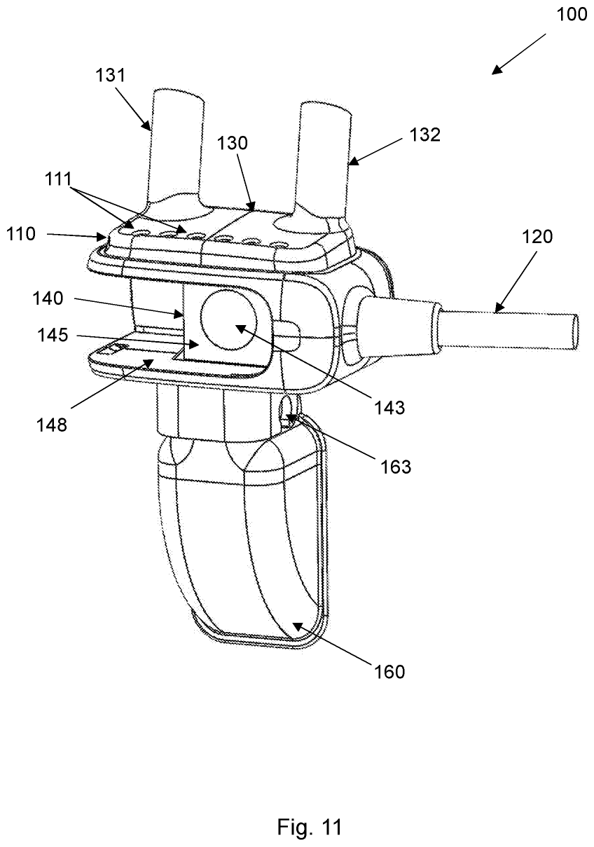

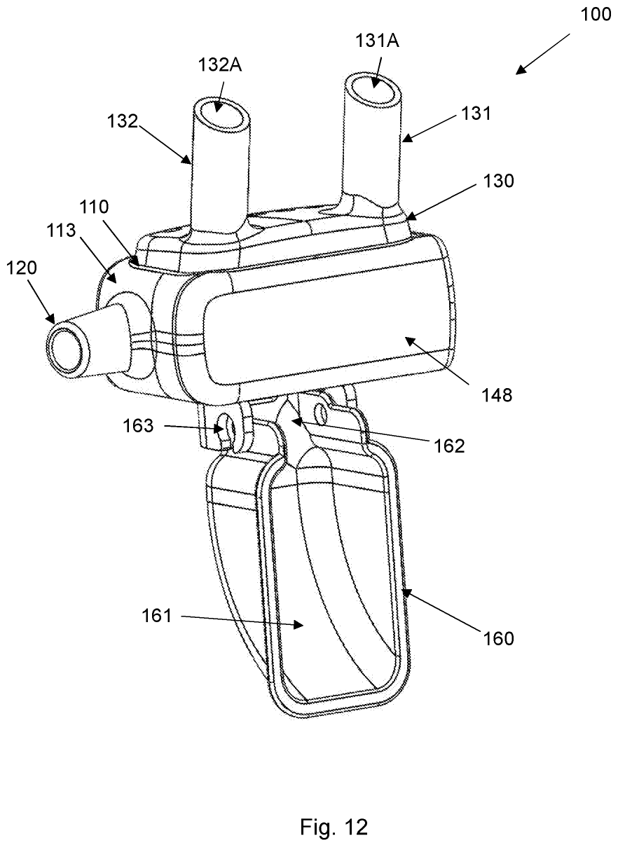

FIGS. 1, 2, 11 and 12 illustrate embodiments of the airway adapter 100 adapted to be disposed below nostrils of a human subject in a front view (FIGS. 1 and 11) and a rear view (FIGS. 2 and 12). The airway adapter 100 comprises a gas case 110, also referred to as gas housing herein, comprising multiple gas outlets 111 arranged to be positioned in vicinity of the nostrils of the human subject when the airway adapter 100 is attached on or to the face of the human subject. The airway adapter 100 also comprises a gas delivery tube 120 in fluid communication, i.e., fluid connection, with the gas case 110. A prong case 130, also referred to as prong housing herein, comprising at least one nasal prong 131, 132 is included in the airway adapter 100 and arranged to collect nasally exhaled breath from the human subject. The airway adapter 100 further comprises an airway case 140, also referred to as airway housing. The airway case 140 defines an airway passage 141 in fluid communication with the prong case 130. The airway case 140 comprises a first light window 142 into the airway passage 141 and a second light window 143 into the airway passage 141. The airway adapter 100 is connectable to a mainstream capnography sensor 200 comprising at least one light source 220 and at least one light detector 230 to position the mainstream capnography sensor 200 onto the airway adapter 100 to align the at least one light source 220 with the first light window 142 and align the at least one light detector 230 with the second light window 143, see FIGS. 4-6, 13-14.

Fluid communication as used herein means that a fluid, and in particular a gas, can flow between two units or devices that are in fluid communication with each other. For instance, the gas delivery tube 120 is in fluid communication with the gas case 110 to thereby enable gas flowing through the gas delivery tube 120 to flow into the gas case 110. Hence, in a particular embodiment, fluid communication as used herein refers to enabling gas to flow between the units or devices. Gas as used herein includes both pure gases of a single element, such as 100% O₂, and gas mixtures, such as air or oxygen-enriched air, i.e., air having a higher oxygen content than 21%.

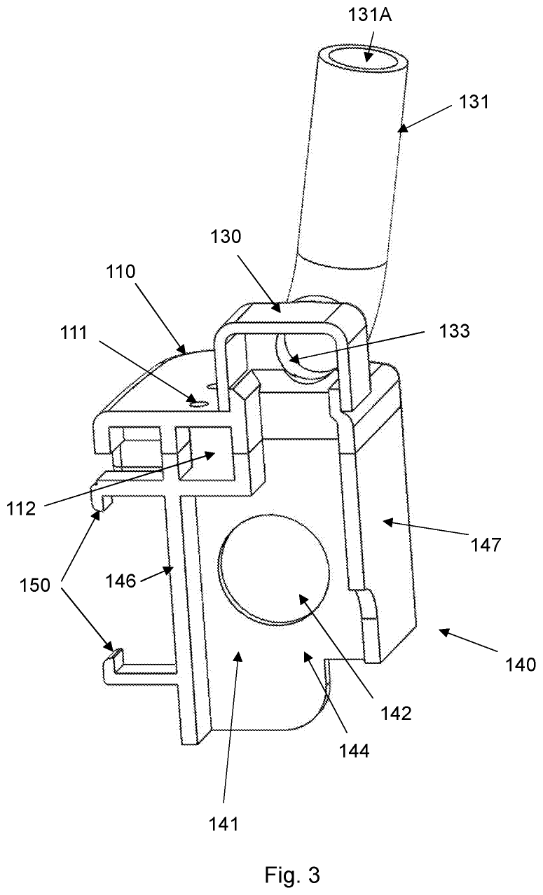

The airway adapter 100 thereby comprises three main cases or housings, the gas case 110, the prong case 130 and the airway case 140. The gas case 110 defines a gas chamber 112 as shown in the cross-sectional view in FIG. 3. The gas case 110 also comprises multiple, i.e., at least two, gas outlets 111, such as in the form of multiple through holes in the gas case 110 and into the gas chamber 112. The gas case 110 is in fluid communication with the gas delivery tube 120. In a typical embodiment, the gas delivery tube 120 is connected to and ends at a side wall of the gas case 110 as shown in FIGS. 1, 2, 11 and 12. Any gas flown through the gas delivery tube 120 will thereby enter the gas case 110 and its gas chamber 112 though an opening in the side wall 113. The delivered gas will flow through the gas chamber 112 and out through the multiple gas outlets 111. Accordingly, the gas flown through the gas delivery tube 120 will thereby be delivered through these multiple gas outlets 111 and reach the human subject as the multiple gas outlets 111 are arranged to be positioned in vicinity of the nostrils of the human subject when the airway adapter 100 is attached on or to the face of the human subject. The gas case 110 could comprise two gas outlets 111, such as one for each of the two nostrils, but typically comprises more than two such gas outlets 111, such as at least three, at least four, or at least five gas outlets 111. In such a case, the multiple gas outlets 111 could be arranged in an array or row as indicated in FIGS. 1 and 11, or be arranged in a matrix or indeed in any pattern for enabling gas delivery from the gas delivery tube 120 into the nostrils.

The prong case 130 comprises at least one nasal prong 131, 132 arranged to collect nasally exhaled breath from the human subject. In an embodiment, the prong case 130 could comprise a single nasal prong arranged to collect nasally exhaled breath from the left nostril or the right nostril. However, in a preferred embodiment, the prong case 130 comprises two nasal prongs 131, 132 as shown in FIGS. 1, 2, 11 and 12, one for each nostril, i.e., a first nasal prong 131 and a second nasal prong 132. The one or two prongs 131, 132 could be in the form of tube(s) extending from the prong case 130 and designed to be at least partly inserted into the nostril(s) or end shortly below the nostrils to thereby enable collection of the nasally exhaled air. Each prong 131, 132 thereby comprises a respective channel 131A, 132A in fluid connection with a prong chamber 133 defined by the prong case 130, see FIG. 3. This means that once the human subject exhales the exhaled air will at least partly flow into the channel(s) 131A, 132A of the nasal prong(s) 131, 132 and flow into the prong chamber 133. The prong case 130 is in fluid communication with the airway case 140 or more correctly the prong chamber 133 of the prong case 130 is in fluid communication with the airway passage 141 defined by the airway case 140. This means that the collected nasally exhaled air will flow through the nasal prong(s) 131, 132, into the prong chamber 133 and then through the airway passage 141. The exhaled air will thereby flow past the two light windows 142, 143 in the airway case 140. The mainstream capnography sensor 200 can then analyze the exhaled air flowing through the airway passage 141 as the human subject is exhaling. This is possible by arranging the at least one light source 220 and the at least light detector 230 of the mainstream capnography sensor 200 in vicinity of and aligned with the light windows 142, 143. Hence, light from the at least one light source 220 is thereby directed through the first light window 142 into the airway passage 141 and through the second light window 143 where the light is detected by the at least one light detector 230, which is further described herein. This means that the two light windows 142, 143 and the portion of the airway passage 141 between the light windows 142, 143 form a gas cuvette, through which exhaled air samples can be analyzed by the mainstream capnography sensor 200.

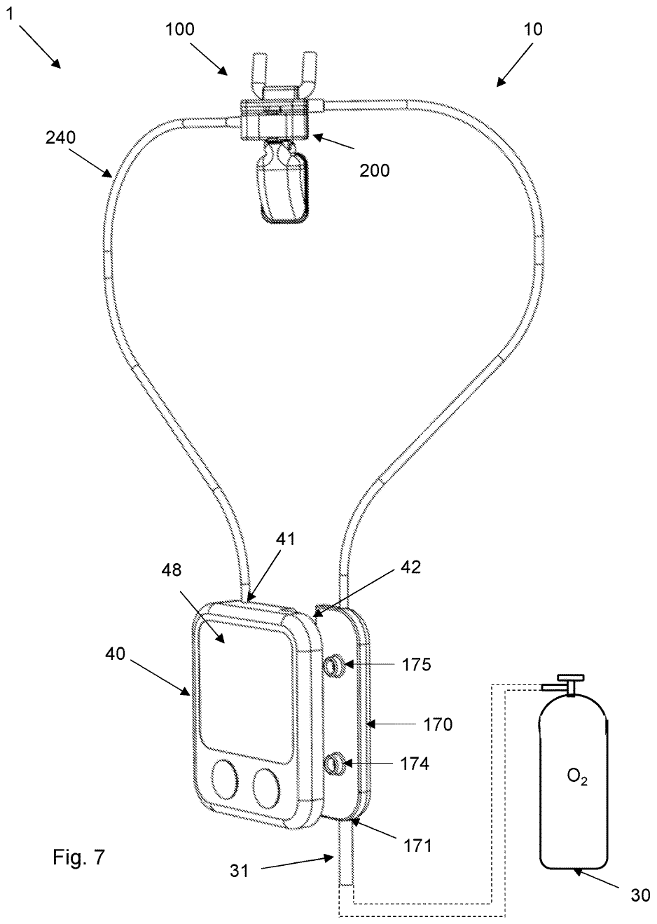

The first and second light windows 142, 143 could be windows made of optically transparent material enabling the light from the light source 220 to pass through the light windows 142, 143. Illustrative, but non-limiting, examples of such materials include optically transparent plastics, glass and sapphire crystal. The gas delivery tube 120 extending from the airway adapter 100, such as from the gas case 110, is adapted to be placed around an ear of the human subject, see FIG. 16. As is more clearly seen in FIG. 7, in the illustrated embodiment, the gas delivery tube 120 is adapted to be placed around the left ear of the human subject. The embodiments are, though, not limited thereto. In another embodiment, the gas delivery tube 120 extends from the opposite side wall 114 of the gas case 110 to thereby be adapted to be placed around the right ear of the human subject. Placing the gas delivery tube 120 around one ear of the human subject and, which is further discussed herein, the sensor cable 240 of the mainstream capnography sensor 200 around the other ear of the human subject will position the airway adapter 100 below the nostrils of the human subject to thereby enable collection of nasally exhaled air through the at least one nasal prong 131, 132 and administer gas through the multiple gas outlets 111 of the gas case 110.

In an embodiment, the gas delivery tube 120 extends acutely from the gas case 110. This is more clearly seen in FIGS. 1, 2, 11 and 12. Hence, in this embodiment, the gas delivery tube 120 extends from the gas case 110 directed towards the face of a human subject rather than extending orthogonally from one of the sides of the gas case 110. Such an acute extension of the gas delivery tube 120 facilitates routing of the gas delivery tube 120 around one of the human subject's ear as the gas delivery tube 120 is thereby extending and being directed towards the ear.

A significant advantage of the airway adapter 100 is that it only needs a single gas delivery tube 120. Hence, in an embodiment, the airway adapter 100 comprises a single gas delivery tube 120 and in particular no other tubes for directing gas from a gas source 30.

This should be compared to prior art airway adapters, such as shown in U.S. Pat. Nos. 7,445,602; 7,455,644; 8,915,861 requiring two gas delivery tubes and two sensor cables giving a total of four tubes or cables. An embodiment of the present invention merely requires a single gas delivery tube 120 and a single sensor cable 240 that are together used to attach the airway adapter 100 and the connected mainstream capnography sensor 200 on or to the face of the human subject by routing the gas delivery tube 120 and the sensor cable 240 around respective ears.

The airway adapter 100 of the invention is thereby much easier to use also for non-medical personnel, including the human subject itself, and attach on the face by having a reduced number of tubes 120 and cables 240 as compared to the prior art solutions exemplified by the above cited U.S. patents.

The airway case 140 comprises at least one passage wall 144, 145, 146, 147 defining the airway passage 141. For instance, the airway passage 141 could comprise four connected passage walls 144, 145, 146, 147 defining and enclosing the airway passage 141. In such an embodiment, the airway passage 141 typically has a quadratic or rectangular cross-section. The embodiments are, though, not limited thereto. For instance, a single cylinder wall could be used to define an airway passage 141 having a circular cross-section.

In the embodiment shown in FIGS. 1-3, the airway case 140 comprises multiple, typically four, passage walls 144, 145, 146, 147 defining the airway passage 141. In such an embodiment, a first passage wall 144 of the multiple passage walls 144, 145, 146, 147 comprises the first light window 142 and a second passage wall 145 of the multiple passage walls 144, 145, 146, 147 comprises the second light window 143. In a preferred embodiment, the first and second passage walls 144, 145 are opposite passage walls 144, 145 facing each other. In such an embodiment, the at least one light source 220 and the at least one light detector 230 will be arranged opposite each other with the airway passage 141 running therebetween.

The first and second passage walls 144, 145 are preferably interconnected by a third passage wall 146 and a fourth passage wall 147. The fourth passage wall 147 is then facing the face of the human subject when the airway adapter 100 is attached on the face of the human subject and the third passage wall 146 is opposite to this fourth passage wall 147, i.e., faces away from the face in the embodiment shown in FIGS. 1-3. In the embodiment, shown in FIGS. 11-13, the first passage wall 144 faces the face of the human subject when the airway adapter 100 is attached on the face of the human subject and the second passage wall 145 is opposite to this first passage wall 144, i.e., faces away from the face. The first and second passage walls 144, 145 are preferably interconnected by a third passage wall 146 and a fourth passage wall 147.

The mainstream capnography sensor 200 is connectable to the airway adapter 100. Any type of connection or attachment solution that allows the mainstream capnography sensor 200 to be attached to the airway adapter 100 could be used. In an embodiment, the airway adapter 100 therefore comprises a connector 150 connectable to the mainstream capnography sensor 200 and arranged to position the mainstream capnography sensor 200 onto the airway adapter 100 to align the at least one light source 220 with the first light window 142 and align the at least one light detector 230 with the second light window 143, see FIGS. 1 and 4.

The connector 150 could, for instance, be attached to the above-mentioned third passage wall 146 of the multiple passage walls 144, 145, 146, 147 facing away from the face of the human subject when wearing the airway adapter 100 as shown in FIGS. 1 and 4.

As an illustrative example, the connector 150 could provide a snap-fit connection of the mainstream capnography sensor 200 onto the airway adapter 100. The connector 150 could, for instance, comprise two opposite snap-fit connectors configured to grip the mainstream capnography sensor 200 when positioned between the opposite snap-fit connectors as shown in FIGS. 1 and 4. Also other connector solutions are possible. For instance, the mainstream capnography sensor 200 could comprise the snap-fit connectors, which are then configured to grip the airway case 140, see FIG. 3.

A further solution is to have a magnet attached to the third passage wall 146 of the airway case 140 with a matching magnet or iron piece in the mainstream capnography sensor 200 to magnetically attach the mainstream capnography sensor 200 to the airway adapter 100. In another embodiment, the mainstream capnography sensor 200 comprises a magnet with an iron piece in the airway adapter 100, such as attached to the third passage wall 146 of the airway case 140.

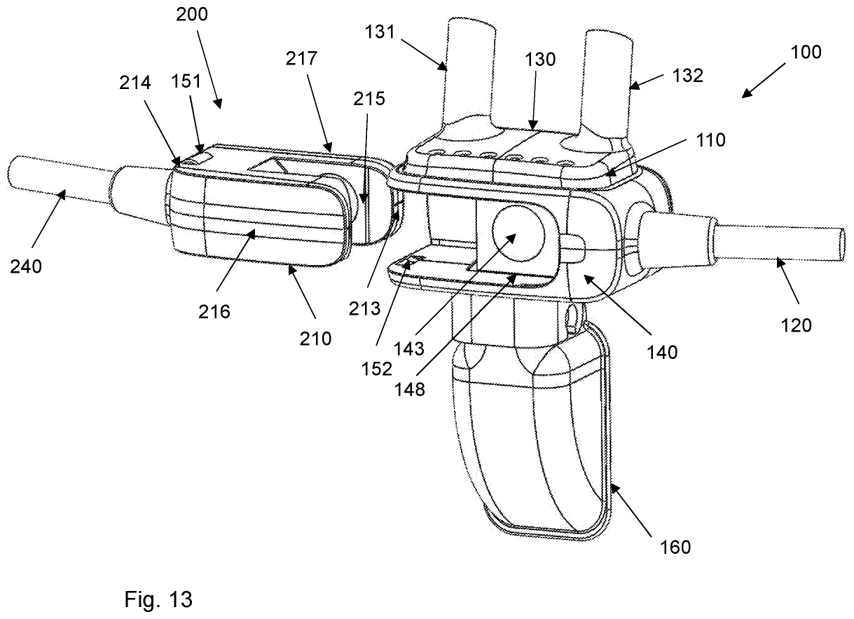

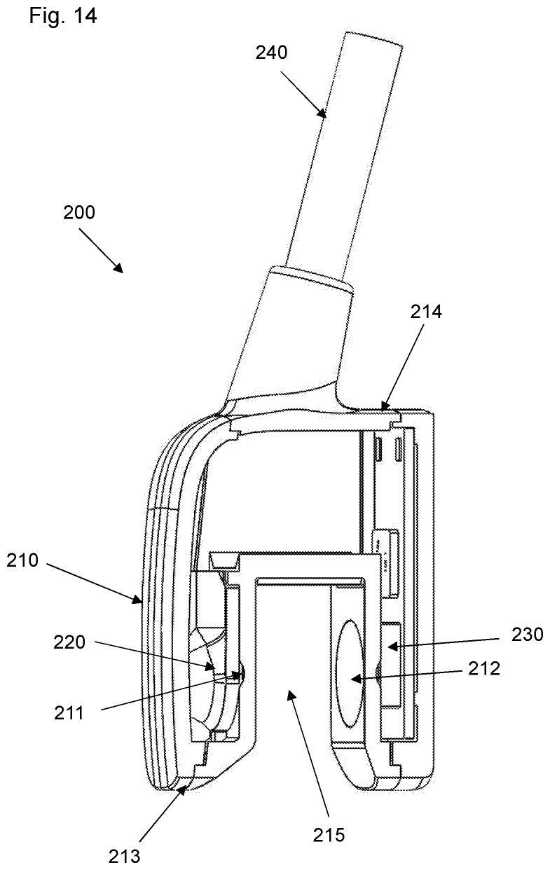

FIGS. 11 and 13 illustrate yet another alternative. In this embodiment, the mainstream capnography sensor 200 comprises a U-shaped sensor housing 210 as best seen in FIG. 14. The sensor housing 210 thereby comprises two legs or arms 216, 217, also referred to as protrusions or extensions herein, each comprising a respective light window 211, 212 and extending from a second short end 214 of the sensor housing 210 to a first short end 213 of the sensor housing 210. The sensor housing 210 thereby has an indentation defined by the extending legs or arms 216, 217. In such an embodiment, the airway case 140 preferably comprises receiving grooves 148 matching the legs or arms 216, 217 of the sensor housing 210 and, which may receive arms 216, 217 when the mainstream capnography sensor 200 is connected to the airway adapter 140 as shown in FIGS. 12 and 13. In such an embodiment, the mainstream capnography sensor 200 could be releasably locked to the airway adapter 100 through a snap-fit connection. Such a snap-fit connection could then be in the form of a protrusion 151 arranged in the sensor housing 210, typically in connection with the second short end 214 and then a matching recess 152 in the airway case 140. When the legs or arms 216, 217 of the sensor housing 210 are fully inserted into the receiving grooves 148 of the airway case 140 the protrusion 151 enters the matching recess 152 and thereby connects the mainstream capnography sensor 200 to the airway adapter 100. In a typical embodiment, two protrusions 151 are arranged on opposite sides in the sensor housing 210 and the airway case 140 then comprises two matching recesses 152. In an alternative embodiment, the one or two matching recesses 152 are instead in the sensor housing 210 with the one or more protrusions 151 present in the airway case 140.

Thus, various mechanical and magnetically connection solutions are possible for the connector 150 of the airway adapter 100 and/or the mainstream capnography sensor 200.

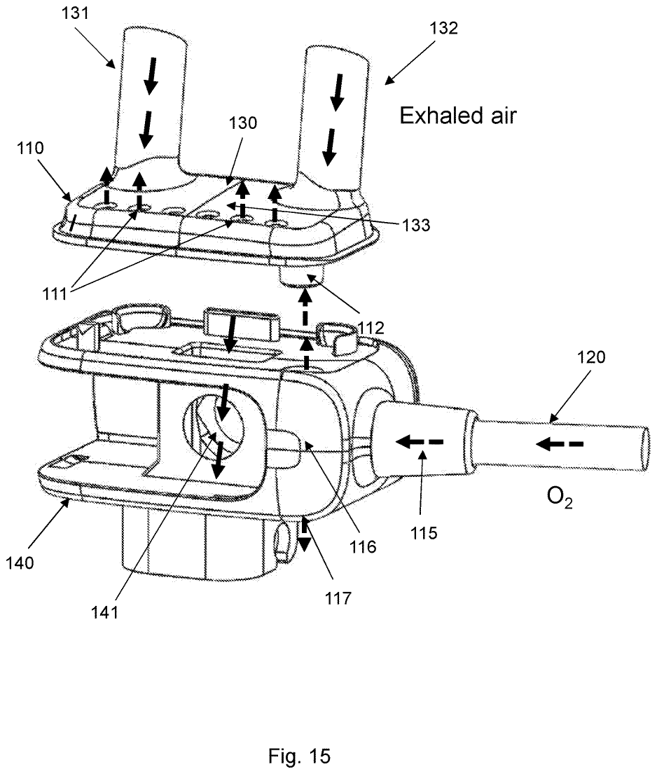

As is shown in FIG. 1, in an embodiment, the prong case 130 is arranged on the gas case 110 to thereby be positioned above the gas case 110 when the airway adapter 100 is attached on the face of the human subject. Correspondingly, in an embodiment, the gas case 110 is arranged on the airway case 140 to thereby be positioned above the airway case 140 when the airway adapter 100 is attached on the face. FIG. 11 illustrates an alternative embodiment, in which the prong case 130 is arranged adjacent or next to the gas case 110. In fact, the prong case 130 and the gas case 110 could arranged as a common case or structure that could be detachable from the airway case 140 as shown in FIG. 15.

FIG. 15 also schematically indicates the flow of gases, i.e., exhaled air and, for instance, oxygen (O₂), through the airway adapter 100. In this illustrative embodiment, oxygen is flowing through the gas delivery tube 210 and into an entrance channel 115 of the airway case 140. The entrance channel 115 is in fluid connection with a guide channel 116 and is connected thereto, for instance, at a T-junction. The oxygen will then flow from the entrance channel 115 into the guide channel 116 where the oxygen flow is diverted into a first or upward flow toward the gas chamber 112 in the gas case 110 and a second or downward flow out through an aperture 117 delivering part of the oxygen flow toward the mouth of the user then the airway adapter 100 is attached to the face of the user. The part of the oxygen flow that is guided into the gas chamber 112 and out through the multiple gas outlets 111.

Correspondingly, exhaled air from the nose of the user is flowing through the at least one nasal prong 131, 132 and into the prong chamber 133 of the prong case 130. The exhaled air is then flowing from the prong chamber 133 into the airway passage 141 of the airway case 140.

The airway adapter 100 may be configured to not only collect nasally exhaled air through the at least one nasal prong 131, 132. In an embodiment, the airway adapter 100 may also comprise a gas guide 160 adapted to be disposed in front of a mouth of the human subject to collect mouth-exhaled breath from the human subject and guide the mouth-exhaled breath into the airway passage 141.

In an embodiment, this gas guide 160 is attached to and extending below the airway case 140. The side 161 of the gas guide 160 facing the mouth of the human subject when the airway adapter 100 is worn is preferably scoop shaped to collect the air exhaled from the mouth. The gas guide 160 also comprises a gas passage 162, such as in the form of a U-shape gas passage 162 that is in fluid communication with the air passage 141. This means that when the human subject exhales by the mouth, the exhaled air hits the side 161 of the gas guide 160 facing the mouth and is guided, by the scoop-shape of this side 161 towards the gas passage 162 and further into the air passage 141. In such an embodiment, both nasally exhaled air and mouth-exhaled air will pass through air passage 141.

The gas guide 160 is preferably detachably connected to the airway adapter 100, and in particular to the airway case 140, and extends below the airway case 140 when the airway adapter 100 is attached on the face of the human subject. Hence, in a preferred embodiment, the attachment 163 of the gas guide 160 to the airway case 140 is a releasable attachment 163 so that the gas guide 160 could be removed from the airway adapter 100 if the human subject would not like to use it to collect mouth-exhaled air but rather only collect nasally-exhaled air by the airway adapter 100.

The attachment 163 may also be a pivotal attachment 163 so that the gas guide 160 is allowed to swing at least slightly relative to the airway case 140. This allows the gas guide 160 to be adjusted close to the mouth of the human subject.

The airway adapter 100 including the gas delivery tube 120 is preferably a disposable airway adapter 100. In such a case, the airway adapter 100 could be used by a human subject and then disposed when no longer needed. This should be compared to the mainstream capnography sensor 200 and the portable monitor 40, to be further described herein, which are preferably reusable. This means that the mainstream capnography sensor 200 and the portable monitor 40 could then be used by different human subjects but each such human subject then has his/her own disposable airway adapter 100. The human subject may also dispose and replace the disposable airway adapter 100 following use thereof for a given period of time.

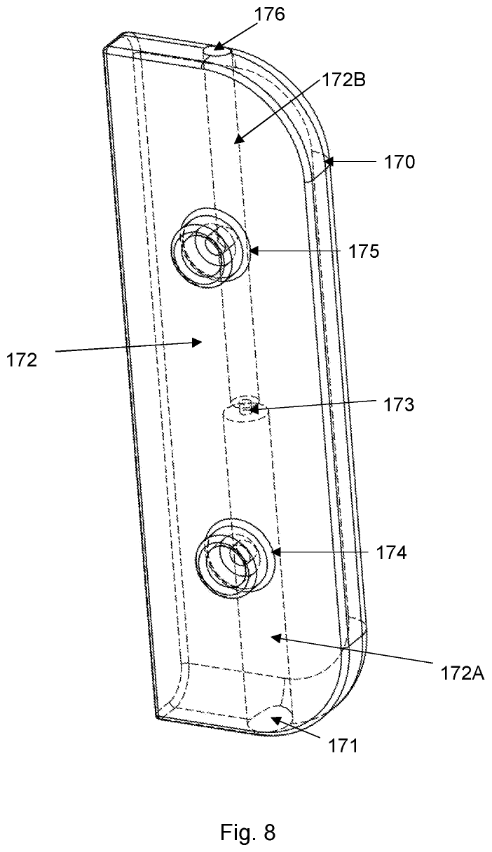

The present invention also relates to an airway adapter arrangement 10, see FIGS. 7 and 8. The airway adapter arrangement 10 comprises an airway adapter 100 according to the invention and a flow measurement adapter 170 attached to the gas delivery tube 120. The flow measurement adapter 170 comprises an inlet port 171 connectable to a gas source 30 through a gas forwarding tube 31. The measurement adapter 170 also comprises a flow channel 172 in fluid communication with the gas delivery tube 120 and the inlet port 171. This flow channel 172 comprises a flow restriction 173. The flow measurement adapter 170 also comprises a first pressure measuring port 174 in fluid communication with the flow channel 172 at a position upstream of the flow restriction 173 and a second pressure measuring port 175 in fluid communication with the flow channel 172 downstream of the flow restriction 173.

Upstream and downstream as used herein with reference to the flow channel 172 is in the direction of the gas flow from the gas source 30 through the flow channel 172, i.e., from the inlet port 171 to an outlet port 176, at which the gas delivery tube 120 is attached to the flow measurement adapter 170.

The flow channel 172 could thereby be regarded as comprising an upstream flow channel 172A extending from the inlet port 171 to the flow restriction 173 and a downstream flow channel 172B extending from the flow restriction 173 to the outlet port 176 and the gas delivery tube 120.

The flow restriction 173 in the flow channel 172 could be any type of structure that restricts the gas flow through the flow channel 172. A typical example of such a flow restriction 173 is to have a channel portion with smaller cross-sectional area, for instance due to smaller diameter, than the cross-sectional area and diameter of the upstream flow channel 172A and the downstream flow channel 172B. The smaller cross-sectional area, such as diameter, at the flow restriction 173 restricts the gas flow through the flow channel 172 to enable measurement of a differential pressure between the upstream and downstream flow channels 172A, 172B. Accordingly, a first or upstream pressure measuring port 174 is in fluid communication with the upstream flow channel 172A and a second or downstream pressure measuring port 175 is in fluid communication with the downstream flow channel 172B.

The gas delivery tube 120 could be detachably attached to the outlet port 176 and could thereby be removed from the flow measurement adapter 170. In another embodiment, the gas delivery tube 120 is fixed to, i.e., permanently attached to, the flow measurement adapter 170 at the outlet port 176 to fluidly connect the gas delivery tube 120 with the flow channel 172. In such a case, the gas delivery tube 120 could be glued or welded to the flow measurement adapter 170.

In an embodiment, the airway adapter arrangement 10 is a disposable airway adapter arrangement 10. Hence, in such an embodiment, not only the airway adapter 100 but also the flow measurement adapter 170 are disposable and could be discarded following use.

The airway adapter 100 and the flow measurement adapter 170 of the airway adapter arrangement 10 are typically made of plastics, including various plastic materials.

The present invention also relates to a capnography and gas delivery arrangement 20, see FIGS. 4-7, 13-14. The capnography and gas delivery arrangement 20 comprises an airway adapter 100 according to the invention or an airway adapter arrangement 10 according to the invention. The capnography and gas delivery arrangement 20 also comprises a mainstream capnography sensor 200. The mainstream capnography sensor 200 comprises a sensor housing 210 comprising a first light window 211 and a second light window 212. The sensor housing 210 also comprises at least one light source 220 arranged in the sensor housing 210 to emit infrared (IR) light through the first light window 211 and at least one light detector 230 arranged in the sensor housing 210 to detect IR light through the second light window 212. The mainstream capnography sensor 200 further comprises a sensor cable 240 attached to the sensor housing 210 and in electrical communication with the at least one light source 220 and the at least one light detector 230.

The first light window 211 of the sensor housing 210 is aligned with the first light window 142 of the airway case 140 and the second light window 212 of the sensor housing 210 is aligned with the second light window 143 of the airway case 140 when the mainstream capnography sensor 200 is attached to the airway adapter 100.

The capnography and gas delivery system 20 thereby comprises the previously described and in FIGS. 1-3, 11-13 shown airway adapter 100, the mainstream capnography sensor 200 and optionally, but preferably also the flow measurement adapter 170.

In an embodiment, the mainstream capnography sensor 200 is attached to the airway adapter 100 as shown in FIG. 7 or 13, such as using the previously described connector 150 of the airway adapter 100 or a connector of the mainstream capnography sensor 200. In this latter case, the mainstream capnography sensor 200 comprises a connector connectable to the airway adapter 100 and arranged to position the mainstream capnography sensor 200 onto the airway adapter 100 to align the first light window 211 of the sensor housing 210 with the first light window 142 of the airway case 140 and align the second light window 212 of the sensor housing 210 with the second light window 143 of the airway adapter 100. In these embodiments, when the mainstream capnography sensor 200 is attached to the airway adapter 100, the first light windows 142, 211 of the airway case 140 and the sensor housing 210 are aligned to each other and the second light windows 143, 212 of the airway case 140 and the sensor housing are likewise aligned to each other. This means that IR light from the at least one light source 220 is then directed through the first light window 211 of the sensor housing 210 and the first light window 142 of the airway case 140 and into the airway passage 141. The emitted IR light will flow through the second light window 143 of the airway case 140 and the second light window 212 of the sensor housing 210 to be captured by the at least one light detector 230.

The light windows 211, 212 of the sensor housing 210 could be windows made of optically transparent material enabling the IR light from the light source 220 to pass through the light windows 211, 212. Illustrative, but non-limiting, examples of such materials include optically transparent plastics, glass and sapphire crystal.

In the embodiment shown in FIGS. 1-7, the light windows 142, 143, 212, 213 of the airway case 140 and of the mainstream capnography sensor 200 are arranged so that IR light emitted from the at least one light source 220 through the airway passage 141 and toward the at least one detector 230 is in a direction from one side of the airway adapter 100 towards the opposite of the airway adapter 100 when positioned at the face of a user. FIGS. 11-14 illustrate an alternative embodiment, in which the light windows 142, 143, 212, 213 of the airway case 140 and of the mainstream capnography sensor 200 are arranged so that IR light emitted from the at least one light source 220 is perpendicularly to the embodiment shown in FIGS. 1-7, i.e., in a direction towards or away from the face of the user then the airway adapter 100 is positioned at the face of a user.

In an embodiment, the gas delivery tube 120 and the sensor cable 240 extend in opposite directions from the gas case 110 and the sensor housing 210 respectively, when the mainstream capnography sensor 200 is connected to the airway adapter 100. Accordingly, the gas delivery tube 120 and the sensor cable 240 are extended to enable one of the gas delivery tube 120 and the sensor cable 240 to be routed around one of the human subject's ear with the other of the gas delivery tube 120 and the sensor cable 240 around the other ear as shown in FIG. 16.

Hence, the gas delivery tube 120 is adapted to be placed around one ear of the human subject and the sensor cable 240 is adapted to be placed around the other ear of the human subject, see FIG. 16.

The gas case 110 comprises a first short end or side 113 and a second, opposite short end or side 114. The sensor housing 210 correspondingly comprises a first short end or side 213 and a second, opposite short end or side 214. The first short ends or sides 113, 213 of the gas case 110 and the sensor housing 210 face the same direction and the second, opposite short ends or sides 114, 214 of the gas case 110 and the sensor housing 210 face the same direction when the mainstream capnography sensor 200 is connected to the airway adapter 100. In such a case, the gas delivery tube 120 extends, preferably acutely, from the first short end or side 113 of the gas case 110 and the sensor cable 240 extends, preferably acutely, from the second short end or side 214 of the sensor housing 210.

The preferred acute extension of the gas delivery tube 120 from the gas case 110 and the preferred acute extension of the sensor cable 240 from the sensor housing 210 facilitate a routing of the gas delivery tube 120 and the sensor cable 240 towards and over the outer ears of the human subject to thereby keep the airway adapter 100 with connected mainstream capnography sensor 200 positioned below the nostrils of the human subject.

As mentioned in the foregoing, the airway adapter 100 preferably comprises a single gas delivery tube 120 to guide the gas from the gas source 30 to the gas case 110 and through the multiple gas outlets 111. Correspondingly, the mainstream capnography sensor 200 preferably comprises a single sensor cable 240 for providing power to the at least one light source 220 and the at least one light detector 230 and forward output signals from the at least one light detector 230 to a portable monitor 40.

In an embodiment, the sensor housing 210 comprises an indentation 215. In such an embodiment, the first and second light windows 211, 212 of the sensor housing 210 face the indentation 215. At least a portion of the airway case 140 is adapted to be positioned in the indentation 215 when the mainstream capnography sensor 200 is connected to the airway adapter 100.

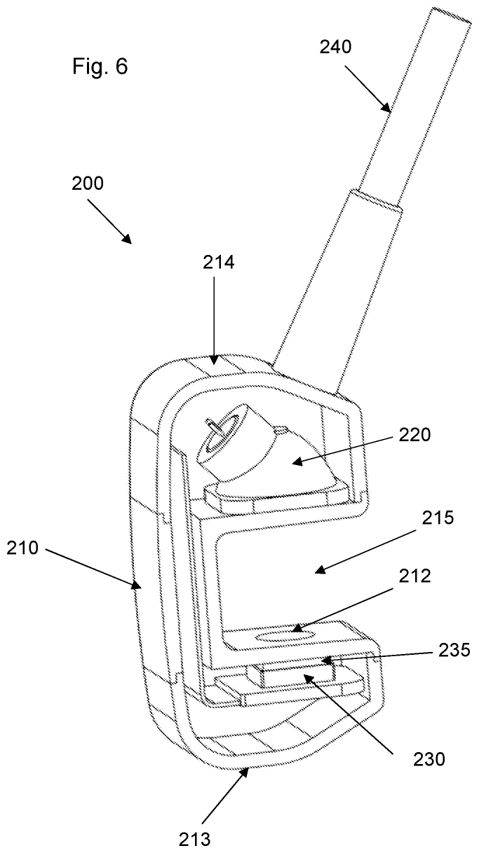

Hence, as shown in FIGS. 6 and 14, the sensor housing 210 of the mainstream capnography sensor 200 has a general U-shape with the two light windows 211, 212 facing the space between the two “arms” or “legs” 216, 217 of the U. In such a case, at least a portion of the airway case 140 is adapted to be positioned in this space so that a portion of the airway passage 141 will be present between the two “arms” or “legs” 216, 217 and the two light windows 211, 212 of the sensor housing 210 will face the intermediate airway passage 141.

In an embodiment, the sensor housing 210 comprises a single light source 220 configured to emit IR light. In another embodiment, the sensor housing 210 comprises multiple light sources 220 configured to emit IR light, typically of different wavelengths or wavelength intervals.

The light source 220 is preferably configured to emit IR light at one or more wavelengths within the spectrum from 3 μm up to 5 μm. In an embodiment, the light source 220 emits IR light of a single wavelength, such 4.3 μm, at multiple separate wavelengths, such at 3.9 μm and 4.3 μm, or at a band or spectrum of wavelengths, such as from 3 μm up to 5 μm.

In an embodiment, the sensor housing 210 comprises a single light detector 230 configured to detect IR light emitted from the at least one light source 220 and having passed through at least a portion of the airway passage 141. In another embodiment, the sensor housing 210 comprises multiple light detectors 230.

In an embodiment, the sensor housing 210 comprises a respective optical bandpass filter 235 interposed between the second light window 212 of the sensor housing 210 and the at least one light detector 230.

In an embodiment, the optical bandpass filter 235 has a center wavelength (CWL) of 4.3 μm and a full-width at half-maximum (FWHM) of about 100 nm. Such an optical bandpass filter 235 is suitable for measurement of CO₂ by the light detector 230.

In another embodiment, the sensor housing 210 of the mainstream capnography sensor 200 comprises the above-mentioned optical bandpass filter 235 (CWL=4.3 μm, FWHM≈100 nm) and a light detector 230 for detection and measurement of CO₂ and a reference light detector 230 and a reference optical bandpass filter for reference light measurements. The reference light detector 230 and reference optical bandpass filter can then be used to compensate for, for instance, drift in the light source 220, moisture or dirt on the light windows 142, 143, 211, 212, etc. In an embodiment, the reference bandpass filter has a CWL of 3.9 μm and a FWHM of about 100 nm.

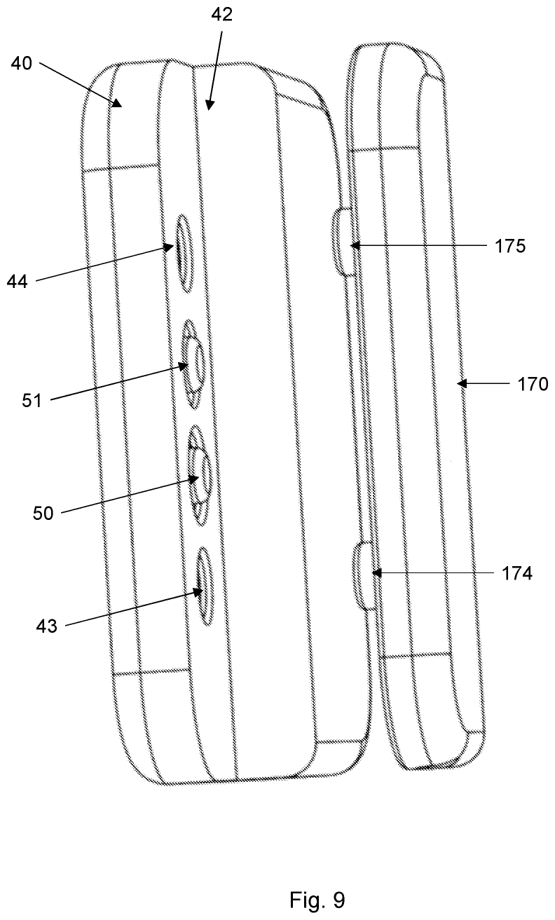

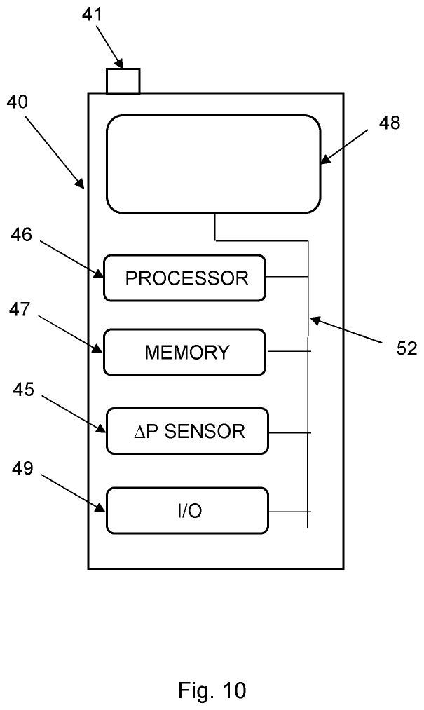

A further aspect of the invention relates to a portable monitor 40 for monitoring of a spontaneously breathing human subject, see FIGS. 7, 9-10. The portable monitor 40 comprises a sensor port 41 connectable to a sensor cable 240 of a mainstream capnography sensor 200. The portable monitor 40 also comprises an adapter receptacle 42 connectable to a flow measurement adapter 170. A first pressure port 43 of the portable monitor 40 is configured to be in fluid connection with a gas flow through the flow measurement adapter 170 upstream of a flow restriction 173 and a second pressure port 44 of the portable monitor 40 is configured to be in fluid communication with the gas flow through the flow measurement adapter 170 downstream of the flow restriction 173. The portable monitor 40 also comprises a differential pressure sensor 45 in fluid communication with the first pressure port 43 and the second pressure port 44. The differential pressure sensor 45 is configured to measure a pressure difference between the first pressure port 43 and the second pressure port 44 and generate an output signal representative of the pressure difference.

The portable monitor 40 also comprises a processor 46 communicatively connected to the sensor port 41 and the differential pressure sensor 45 and a memory 47 coupled to the processor 46. The memory 47 comprises instructions executable by the processor 46 to cause the processor 46 to process an output signal received at the sensor port 41 and generated by the mainstream capnography sensor 200 to generate a CO₂ parameter value representative of partial pressure of CO₂ in the exhaled air from the human subject. The processor 46 is also caused to process the output signal from the differential pressure sensor 45 to generate a flow rate value representative of a flow rate of the gas flow.

The portable monitor 40 is thereby a portable device, i.e., handheld or more preferably hanging attached to the gas delivery tube 120 and the sensor cable 240 as shown in FIG. 7, which can be used together with the mainstream capnography sensor 200 to monitor CO₂ partial pressure in the air exhaled by the human subject at the same time as the portable monitor 40 is able to monitor the gas flow from the gas source 30 to the airway adapter 100 and to be inhaled by the human subject.

The portable monitor 40 can thereby be used as a diagnostic tool for patient monitoring and in particular when there is a need to monitor partial pressure of CO₂ at the same time as delivering gas, such as oxygen or oxygen-enriched air, to the human subject.

In an embodiment, the memory 47 comprises instructions executable by the processor 46 to cause the processor 46 to process the output signal received at the sensor port 41 to generate a respiratory rate (RR) value representative of a respiratory rate of the human subject. Hence, the output signal from the mainstream capnography sensor 200 can not only be used to determine the partial pressure of CO₂ but also for estimating the respiratory rate of the human subject.

In an embodiment, the memory 47 comprises an RR high threshold value and/or an RR low threshold value. The memory 47 also comprises, in this embodiment, instructions executable by the processor 46 to cause the processor 46 to compare the RR value with the RR high threshold value and/or the RR low threshold value. The processor 46 is also caused to generate an RR high alarm signal if the RR value exceeds the RR high threshold value or an RR low alarm signal if the RR value is below the RR low threshold.

The respiratory rate is typically determined by the processor 46 based on the time interval between subsequent EtCO₂ in the CO₂ waveform or signal.

The respiratory rate for a healthy adult human subject is typically within a range of from 12 up to 18 breaths per minute (bpm). Hence, the RR low threshold value is preferably set to be below 12, such as 10 or 8 bpm, whereas the RR high threshold value is preferably set to a value above 18, such as 22 or 24 bpm.

The portable monitor 40 may, thus, be used to monitor the respiratory rate of the human subject to verify that the respiratory rate is within acceptable limits as defined by the RR high and low threshold values. A low respiratory rate, also referred to as bradypnea, can occur as a result of a worsening of an underlying respiratory condition, in connection with cardiac arrest, drug overdose or obstructive sleep apnea. A high respiratory rate, also referred to as tachypnea, can occur as a result of anxiety, fever, various respiratory conditions, and heat problems.

The portable monitor 40 can thereby alarm the human subject or other human subjects in the vicinity of any too high or too low respiratory rates.

In an embodiment, the memory 47 comprises instructions executable by the processor 46 to cause the processor 46 to determine an estimate of inspired gas concentration based on the RR value and the flow rate value. Hence, the portable monitor 40 is capable of estimating the inspired gas concentration based on the determined RR value and the flow rate value. This inspired gas concentration is an important parameter to verify that the human subject is inspiring correct gas concentration.

In an embodiment, the memory 47 comprises information, such as in the form of a look-up table, comprising measured inspired gas concentration as a function of respiratory rate and flow rate. In such an embodiment, the processor 46 is configured to retrieve an estimate of the inspired gas concentration based on the determined RR value and the flow rate value from the look-up table. In other embodiment, the processor 46 is configured to calculate the inspired gas concentration based on a pre-defined function (f( )), such as stored in the memory 47, outputting estimates of inspired gas concentration (Fᵢ) based on input RR values (RR) and flow rate values (Q), i.e., Fᵢ=f(RR, Q).

In an embodiment, the memory 47 comprises a gas concentration high threshold value and/or a gas concentration low threshold value. The memory 47 also comprises instructions executable by the processor 46 to cause the processor 46 to compare the estimate of inspired gas concentration with the gas concentration high threshold value and/or the gas concentration low threshold value. The processor 46 is also caused to generate a gas high alarm signal if the estimate of inspired gas concentration exceeds the gas concentration high threshold value or a gas low alarm signal if the estimate of inspired gas concentration is below the gas concentration low threshold.

The monitoring of the inspired gas concentration could be used to verify that the gas flow from the gas source 30 delivers sufficient gas to be inspired by human subject given his/her current respiration rate. In other words, if there is a mismatch between the respiration rate and the flow rate of the gas, then the inspired gas concentration might be higher or lower than a target inspired gas concentration as prescribed by a physician or other medical personnel. The portable monitor 40 may thereby signal an alarm if such a condition is detected to notify that there is a need to adjust the flow rate of gas from the gas source 30 to match the human subject's current respiratory rate.

In an embodiment, the memory 47 comprises instructions executable by the processor 46 to cause the processor 46 to process the output signal received at the sensor port 41 and generated by the mainstream capnography sensor 200 to generate an end-tidal CO₂ (ETCO₂) value.

EtCO₂ monitoring is a non-invasive technique that measures the partial pressure of maximum concentration of CO₂ at the end of an exhaled breath. EtCO₂ is typically expressed as a percentage of CO₂ or mmHg. Normal values for EtCO₂ are 5 to 6% CO₂, which corresponds to 35-45 mmHg at sea level.

The processor 46 is typically configured to identify a local maximum in the CO₂ waveform or signal corresponding to the end of an expiration. This local maximum is followed by a sharp drop in the CO₂ waveform or signal corresponding to an inspiration. The local maximum in the CO₂ waveform or signal corresponds to the EtCO₂.

In an embodiment, the memory 47 comprises instructions executable by the processor 46 to cause the processor 46 to determine a trend in the EtCO₂ value during a measurement period. In this embodiment, the processor 46 is also caused to generate an alarm signal, such as a hypercapnia alarm signal, if the EtCO₂ value increases with at least a predefined percentage during the measurement period and if the flow rate value represents a non-zero flow rate of the gas flow during the measurement period.

Hence, in this embodiment, the portable monitor 40 can use the output signals from the mainstream capnography sensor 200 and the differential pressure sensor 45 to detect a potential episode of hypercapnia of the human subject. Thus, if the flow rate value as determined by the processor 46 based on the output signal from the differential pressure sensor 45 represents a non-zero value, i.e., there is a gas flow from the gas source 30 to the airway adapter 100 but the output signal from the mainstream capnography sensor 200, as processed by the processor 46, indicates an increase in EtCO₂ during a measurement period then the human subject is likely to suffer from hypercapnia or may at least have a risk of suffering from hypercapnia.

In an embodiment, the memory 47 comprises an EtCO₂ high threshold value and/or an EtCO₂ low threshold value. In this embodiment, the memory 47 comprises instructions executable by the processor 46 to cause the processor 46 to compare the EtCO₂ value with the EtCO₂ high threshold value and/or the EtCO₂ low threshold value. In this embodiment, the processor 46 is also caused to generate an EtCO₂ high alarm signal if the EtCO₂ value exceeds the EtCO₂ high threshold value or an EtCO₂ low alarm signal if the EtCO₂ value is below the EtCO₂ low threshold.

Illustrative, but non-limiting, examples of the EtCO₂ high and low threshold values include 50 mmHg and 0-30 mmHg.

In an embodiment, the memory 47 comprises a flow rate high threshold value and/or a flow rate low threshold value. The memory 47 also comprises instructions executable by the processor 46 to cause the processor 46 to compare the flow rate value with the flow rate high threshold value and/or the flow rate low threshold value. In this embodiment, the processor 46 is also caused to generate a flow rate high alarm signal if the flow rate value exceeds the flow rate high threshold value or a flow rate low alarm signal if the flow rate value is below the flow rate low threshold.

In an embodiment, the portable monitor 40 also comprises a display screen 48 communicatively connected to the processor 46. In this embodiment, the memory 47 comprises instructions executable by the processor 46 to cause the processor 46 to display the CO₂ parameter value and/or waveform on the display screen 48 and display the gas flow value on the display screen 48.

The processor 46 may be also caused to display the above-described diagnostic parameters including RR value, inspired gas concentration, and/or ETCO₂ value.

The display screen 48 could display the one or more diagnostic parameters as single value and/or as a trend, such as a graph showing how the values of the one or more diagnostic parameters change over time.

The above-described alarms could be visible alarms presented on the display screen 48 and/or audible alarms.

In an embodiment, the portable monitor 40 comprises a communication unit 49 communicatively connected to the processor 46. In such an embodiment, the memory 47 comprises instructions executable by the processor 46 to cause the processor 46 to instruct the communication unit 49 to transmit the CO₂ parameter value or information representative of the CO₂ parameter value to an external device and instruct the communication unit 49 to transmit the gas flow value or information representative of the gas flow value to the external device.

The processor 46 may also cause the communication unit 49 to transmit the above-described diagnostic parameters including RR value, inspired gas concentration, and/or ETCO₂ value to the external device.

The communication unit 49 may be a transmitter or transceiver configured to conduct wireless communication with the external device. In another embodiment, the communication unit 49 is an output port or a combined input and output (I/O) port for wired communication with the external device.

The external device could be a mobile phone, table or computer of the human subject or a computer or computer system of a physician or healthcare facility as illustrative but non-limiting examples.

The adapter receptacle 42 of the portable monitor 40 is connectable to the flow measurement adapter 170. In an embodiment, a switch 50 of the portable monitor 40 is arranged in connection with the adapter receptacle 42 to indicate when the flow measurement adapter 170 is connected to the adapter receptacle 42. In such an embodiment, the processor 46 is responsive to the output signal from the switch 50 and will then process the output signal from the differential pressure sensor 45 if, and preferably only if, the switch 50 indicates that the flow measurement adapter 170 is connected to the adapter receptacle 42 of the portable monitor 40. In such a case, the differential pressure measurements will only take place when the portable sensor 40 is connected to the flow measurement adapter 170 and the gas flow through the flow channel 172 of the flow measurement adapter 170.

In an embodiment, the portable monitor 40 comprises a second switch 51. In such an embodiment, this second switch 51 could generate an output signal when an adapter 170 is connected to the adapter receptacle 42. The output signal from the second switch 51 is then indicative of the type of adapter 170 that is connected to the adapter receptacle 42. For instance, the adapter receptacle 42 of the portable monitor 40 could be configured to be connected to different types of adapters 170. As an example, a first flow measurement adapter 170 is to be used in connection with adult users and a second flow measurement adapter 170 is to be used in connection with children. Another example is to have the option of connecting either a flow measurement adapter 170 or an adapter for pressure measurements to the adapter receptacle 42. In this latter case, the adapter for pressure measurements could be used to monitor the respiration of the user.

The output signal from the second switch 51 can then be processed by the processor 46 to determine which particular adapter 170 that is connected to the adapter receptacle 42 and can thereby process the output signal from the differential pressure sensor 45 at least partly based on the type of adapter 170 that is currently connected to the adapter receptacle 42 as indicated by the output signal from the second switch 51.

The first pressure port 43 of the portable monitor 40 is in fluid communication with the first pressure measuring port 174 of the flow measurement adapter 170 and the second pressure port 44 of the portable monitor 40 is in fluid communication with the second pressure measuring port 175 of the flow measurement adapter 170 when the flow measurement adapter 170 is connected to the adapter receptacle 42. In such a case, the differential pressure sensor 45 is able to measure a pressure difference between the first and second pressure ports 43, 44 and thereby between the first and second pressure measuring ports 174, 175 and between the upstream flow channel 172A and the downstream flow channel 172B.

The processor 46 and the memory 47 are communicatively connected to each other. For instance, the portable monitor 40 could comprise a communication bus 52 allowing communication and data transfer between the processor 46 and the memory 47. Also the differential pressure sensor 45, the communication unit 49 and the display screen 48 could be connected to this communication bus 52.

The term processor should be interpreted in a general sense as any circuitry, system or device capable of executing program code or computer program instructions to perform a particular processing, determining or computing task. The processor 46 does not have to be dedicated to only execute the above-described steps, functions, procedure and/or blocks, but may also execute other tasks.

The present invention additionally relates to a mainstream capnography system 1, see FIG. 7. The mainstream capnography system 1 comprises a capnography and gas delivery arrangement 20 according to the invention and comprising an airway adapter arrangement 10 according to the invention, and a portable monitor 40 according to the invention. The mainstream capnography system 1 thereby comprises the airway adapter 100, the flow measurement adapter 170, the mainstream capnography sensor 200 and the portable monitor 40.

During use of the mainstream capnography system 1, the flow measurement adapter 170 is attached to the adapter receptable 42 of the portable monitor 40, the mainstream capnography sensor 200 is connected to the airway adapter 100 and the sensor cable 240 is connected to the sensor port 41 of the portable monitor 40.

In an embodiment, the mainstream capnography system 1 also comprises a gas source 30 in fluid communication with the airway adapter arrangement 10 by a gas forwarding tube 31. The gas forwarding tube 31 is then preferably detachably connectable to the inlet port 171 of the flow measurement adapter 170, or could be permanently attached thereto, such as glued or welded.

The gas source 30 could be any source containing a gas to be administered to the human subject. The gas source 30 is typically a pressurized gas source 30 containing the gas at a higher pressure than ambient pressure (1 bar). For instance, the gas source 30 could be a gas tank or cylinder.

Gas as used herein includes both pure gases of a single element, such as 100% O₂, and gas mixtures, such as air or oxygen-enriched air, i.e., air having a higher oxygen content than 21%.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1-34. (canceled)

35. An airway adapter adapted to be disposed below nostrils of a human subject and comprising:
a gas case comprising multiple gas outlets arranged to be positioned in vicinity of the nostrils of the human subject when the airway adapter is attached on a face of the human subject;
a gas delivery tube in fluid communication with the gas case;
a prong case comprising at least one nasal prong arranged to collect nasally exhaled breath from the human subject; and
an airway case defining an airway passage in fluid communication with the prong case, wherein the airway case comprises:
a first light window into the airway passage; and
a second light window into the airway passage, wherein the airway adapter is connectable to a mainstream capnography sensor comprising at least one light source and at least one light detector to position the mainstream capnography sensor onto the airway adapter to align the at last one light source with the first light window and align the at least one light detector with the second light window.

36. The airway adapter according to claim 35, wherein the gas delivery tube is adapted to be placed around an ear of the human subject.

37. The airway adapter according to claim 35, wherein the gas delivery tube extends acutely from the gas case.

38. The airway adapter according to claim 35, wherein the airway adapter comprises a single gas delivery tube.

39. The airway adapter according to claim 35, wherein with the prong case comprises a first nasal prong and a second nasal prong arranged to collect nasally exhaled breath from the human subject.

40. The airway adapter according to claim 35, wherein
the airway case comprises multiple passage walls defining the airway passage;
a first passage wall of the multiple passage walls comprises the first light window; and
a second, opposite passage wall of the multiple passage walls comprises the second light window.

41. The airway adapter according to claim 35, further comprising a connector connectable to the mainstream capnography sensor and arranged to position the mainstream capnography sensor onto the airway adapter to align the at last one light source with the first light window and align the at least one light detector with the second light window.

42. The airway adapter according to claim 35, further comprising receiving grooves adapted to receive matching arms of the mainstream capnography sensor to position the mainstream capnography sensor onto the airway adapter to align the at last one light source arranged in one matching arm with the first light window and align the at least one light detector arranged in another matching arm with the second light window.

43. The airway adapter according to claim 35, further comprising a gas guide adapted to be disposed in front of a mouth of the human subject to collect mouth-exhaled breath from the human subject and guide the mouth-exhale breath into the airway passage.

44. The airway adapter according to claim 43, wherein the gas guide is detachably attached to the airway case and extends below the airway case when the airway adapter is attached on the face of the human subject.

45. The airway adapter according to claim 35, wherein the airway adapter is a disposable airway adapter.

46. An airway adapter arrangement comprising:
the airway adapter according to claim 35; and
a flow measurement adapter attached to the gas delivery tube and comprising:
an inlet port connectable to a gas source through a gas forwarding tube;
a flow channel in fluid communication with the gas delivery tube and the inlet port and comprising a flow restriction;
a first pressure measuring port in fluid communication with the flow channel at a position upstream of the flow restriction; and
a second pressure measuring port in fluid communication with the flow channel downstream of the flow restriction.

47. The airway adapter arrangement according to claim 46, wherein the airway adapter arrangement is a disposable airway adapter arrangement.

48. A capnography and gas delivery arrangement comprising:
the airway adapter according to claim 35;
a mainstream capnography sensor comprising:
a sensor housing comprising:
a first light window;
a second light window;
at least one light source arranged in the sensor housing to emit infrared (IR) light through the first light window; and
at least one light detector arranged in the sensor housing to detect IR light through the second light window; and
a sensor cable attached to the sensor housing and in electrical communication with the at least one light source and the at least one light detector, wherein
the first light window of the sensor housing is aligned with the first light window of the airway case and the second light window of the sensor housing is aligned with the second light window of the airway case when the mainstream capnography sensor is connected to the airway adapter.

49. The capnography and gas delivery arrangement according to claim 48, wherein the gas delivery tube and the sensor cable extends in opposite directions from the gas case and the sensor housing, respectively, when the mainstream capnography sensor is connected to the airway adapter.

50. The capnography and gas delivery arrangement according to claim 48, wherein
the gas delivery tube is adapted to be placed around one ear of the human subject; and
the sensor cable is adapted to be placed around the other ear of the human subject.

51. The capnography and gas delivery arrangement according to claim 48, wherein
the gas case comprises a first short end and a second, opposite short end;
the sensor housing comprises a first short end and a second, opposite short end;
the gas delivery tube extends acutely from the first short end of the gas case;
the sensor cable extends acutely from the second, opposite short end of the sensor housing; and
the first short ends of the gas case and the sensor housing face the same direction and the second, opposite short ends of the gas case and the sensor housing face the same direction when the mainstream capnography sensor is connected to the airway adapter.

52. The capnography and gas delivery arrangement according to claim 48, wherein
the airway adapter comprises a single gas delivery tube; and
the mainstream capnography sensor comprises a single sensor cable.

53. The capnography and gas delivery arrangement according to claim 48, wherein
the sensor housing comprises an indentation;
the first and second light windows of the sensor housing face the indentation; and
at least a portion of the airway case is adapted to be positioned in the indentation when the mainstream capnography sensor is connected to the airway adapter.

54. The capnography and gas delivery arrangement according to claim 48, wherein the sensor housing comprises a respective optical bandpass filter interposed between the second light window of the sensor housing and the at least one light detector.

55. The capnography and gas delivery arrangement according to claim 48, wherein the mainstream capnography sensor comprises a connector connectable to the airway adapter and arranged to position the mainstream capnography sensor onto the airway adapter to align the first light window of the sensor housing with the first light window of the airway case and align the second light window of the sensor housing with the second light window of the airway case.

56. The capnography and gas delivery arrangement according to claim 48, wherein
the sensor housing is a U-shaped sensor housing comprising two arms;
the first light window and the at least one light source are arranged in a first arm of the two arms;
the second light window and the at least one light detector are arranged in a second arm of the two arms; and
the airway case comprises receiving grooves arranged to receive the two arms to position the mainstream capnography sensor onto the airway adapter to align the first light window of the sensor housing with the first light window of the airway case and align the second light window of the sensor housing with the second light window of the airway case.

57. A portable monitor for monitoring a spontaneously breathing human subject, wherein the portable monitor comprises:
a sensor port connectable to a sensor cable of a mainstream capnography sensor;
an adapter receptacle connectable to a flow measurement adapter;
a first pressure port configured to be in fluid communication with a gas flow through the flow measurement adapter upstream of a flow restriction;
a second pressure port configured to be in fluid communication with the gas flow through the flow measurement adapter downstream of the flow restriction;
a differential pressure sensor in fluid communication with the first pressure port and the second pressure port and configured to measure a pressure difference between the first pressure port and the second pressure port and generate an output signal representative of the pressure difference;
a processor communicatively connected to the sensor port and the differential pressure sensor; and
a memory coupled to the processor and comprising instructions executable by the processor to cause the processor to:
process an output signal received at the sensor port and generated by the mainstream capnography sensor to generate a CO₂ parameter value representative of partial pressure of CO₂ in the exhaled air from the human subject; and
process the output signal from the differential pressure sensor to generate a flow rate value representative of a flow rate of the gas flow.

58. The portable monitor according to claim 57, wherein the memory comprises instructions executable by the processor to cause the processor to process the output signal received at the sensor port to generate a respiratory rate (RR) value representative of a respiratory rate of the human subject.

59. The portable monitor according to claim 58, wherein the memory comprises instructions executable by the processor to cause the processor to determine an estimate of inspired gas concentration based on the RR value and the flow rate value.

60. The portable monitor according to claim 59, wherein the memory comprises a gas concentration high threshold value and/or a gas concentration low threshold value, and instructions executable by the processor to cause the processor to:
compare the estimate of inspired gas concentration with the gas concentration high threshold value and/or the gas concentration low threshold value; and
generate a gas high alarm signal if the estimate of inspired gas concentration exceeds the gas concentration high threshold value or a gas low alarm signal if the estimate of inspired gas concentration is below the gas concentration low threshold.

61. The portable monitor according to claim 57, wherein the memory comprises instructions executable by the processor to cause the processor to process the output signal received at the sensor port and generated by the mainstream capnography sensor to generate an end-tidal CO₂ (ETCO₂) value.

62. The portable monitor according to claim 61, wherein the memory comprises instructions executable by the processor to cause the processor to:
determine a trend in the ETCO₂ value during a measurement period; and
generate an alarm signal if the ETCO₂ value increases with at least a predefined percentage during the measurement period and if the flow rate value represents a non-zero flow rate of the gas flow during the measurement period.

63. The portable monitor according to claim 57, further comprising a display screen communicatively connected to the processor, wherein the memory comprises instructions executable by the processor to cause the processor to:
display the CO₂ parameter value on the display screen; and
display the gas flow value on the display screen.

64. The portable monitor according to claim 57, further comprising a communication unit communicatively connected to the processor, wherein the memory comprises instructions executable by the processor to cause the processor to:
instruct the communication unit to transmit the CO₂ parameter value or information representative of the CO₂ parameter value to an external device; and
instruct the communication unit to transmit the gas flow value or information representative of the gas flow value to the external device.

65. The portable monitor according to claim 57, further comprising a switch arranged in connection with the adapter receptacle and configured to generate an output signal when the flow measurement adapter is connected to the adapter receptacle, wherein the processor is communicatively connected to the switch and is configured to process the output signal from the differential pressure sensor if the output signal from the switch indicates that the flow measurement adapter is connected to the adapter receptacle.

66. A mainstream capnography system comprising:
the capnography and gas delivery arrangement according to claim 48 and further comprising:
a flow measurement adapter attached to the gas delivery tube and comprising:
an inlet port connectable to a gas source through a gas forwarding tube;
a flow channel in fluid communication with the gas delivery tube and the inlet port and comprising a flow restriction;
a first pressure measuring port in fluid communication with the flow channel at a position upstream of the flow restriction; and
a second pressure measuring port in fluid communication with the flow channel downstream of the flow restriction.
; and
a portable monitor comprises:
a sensor port connectable to a sensor cable of a mainstream capnography sensor;
an adapter receptacle connectable to a flow measurement adapter;
a first pressure port configured to be in fluid communication with a gas flow through the flow measurement adapter upstream of a flow restriction;
a second pressure port configured to be in fluid communication with the gas flow through the flow measurement adapter downstream of the flow restriction;
a differential pressure sensor in fluid communication with the first pressure port and the second pressure port and configured to measure a pressure difference between the first pressure port and the second pressure port and generate an output signal representative of the pressure difference;
a processor communicatively connected to the sensor port and the differential pressure sensor; and
a memory coupled to the processor and comprising instructions executable by the processor to cause the processor to:
process an output signal received at the sensor port and generated by the mainstream capnography sensor to generate a CO₂ parameter value representative of partial pressure of CO₂ in the exhaled air from the human subject; and
process the output signal from the differential pressure sensor to generate a flow rate value representative of a flow rate of the gas flow.

67. The mainstream capnography system according to claim 66, wherein the flow measurement adapter is attached to the adapter receptable.

68. The mainstream capnography system according to claim 66, further comprising a gas source in fluid communication with the airway adapter arrangement by a gas forwarding tube.
